(19) United States
(12) Patent Application Publication
(10) Pub. No.: US 2022/0357298 A1
(43) Pub. Date: Nov. 10, 2022

(54) SENSOR ARRANGEMENT AND METHOD OF PUTTING SUCH AN ARRANGEMENT INTO OPERATION

(71) Applicant: Endress+Hauser Conducta GmbH+Co. KG, Gerlingen (DE)

(72) Inventors: Michael Hanko, Dresden (DE); Christian Fanselow, Geringswalde (DE); Hartmut Prinz, Waldheim (DE); Tobias Gühne, Kriebstein (DE)

(21) Appl. No.: 17/662,834

(22) Filed: May 10, 2022

(30) Foreign Application Priority Data
May 10, 2021 (DE) 10 2021 112 184.1

(51) Int. Cl. G01N 27/416; G01N 27/333

(52) U.S. Cl. CPC G01N 27/416; G01N 27/333

(57) ABSTRACT

A sensor arrangement includes a storage chamber comprising an interior space, containing a liquid, with an opening, a reference terminal lead which contacts liquid and can be connected to a superordinate unit, and a sensor tube comprising a sensitive region for detecting a measured quantity of the measuring medium and a measuring terminal lead. The sensitive region can be electrically connected to the superordinate unit. The sensor tube can be moved from a first position into a second position. The sensitive region is located in the interior space of the storage chamber in a first position and outside the storage chamber in the second position. The storage chamber, the opening, and the sensor tube, in the first position, are configured such that the reference/storage/calibration liquid is prevented from escaping from the interior space, and, in the second position, configured such a liquid transport is formed.

### CROSS-REFERENCE TO RELATED APPLICATION

The present application is related to and claims the priority benefit of German Patent Application No. 10 2021 112 184.1, filed on May 10, 2021, the entire contents of which are incorporated herein by reference.

### TECHNICAL FIELD

The present disclosure relates to a sensor arrangement and to a method for putting a sensor arrangement into operation.

### BACKGROUND

Pharmaceutical, biological, biochemical, or biotechnological processes are increasingly performed using what are known as disposable process solutions, for example in process plants in single-use technology. Such process plants comprise pipelines or reactors that are designed as disposable containers (also: disposables or disposable bioreactors or single-use bioreactor or single-use component). These disposable containers can, for example, be flexible containers, for example bags, hoses, or fermenters. Bioreactors or fermenters frequently have supply and discharge lines which are designed as tubes, for example. Stiff pipe sections may also be used in the supply and discharge lines. After a process has ended, the disposable containers may be disposed of. In this way, extensive cleaning and sterilization processes are avoided. The risk of cross-contaminations is prevented, and thus the process reliability is increased, via the use of disposable containers.

In order to monitor or control the processes, it may be necessary to measure physical or chemical measured quantities of the media contained in the disposable process containers. Optical but also electrochemical, such as potentiometric or amperometric, sensors or conductivity sensors are hereby used.

The processes performed in the disposable containers run in a closed system, i.e., without connection to the environment outside the disposable containers. Since sterile conditions are frequently required, the disposable containers must be sterilized prior to introducing the process media. For this purpose, ionizing rays, such as gamma radiation, is often used in biochemical, biological, biotechnological, and pharmaceutical applications. While the processes run in a disposable container, for instance a disposable fermenter or a disposable reactor, the penetration of foreign substances, such as germs, from the environment into the interior of the disposable container must also be avoided in order to not impair or adulterate the process workflow. The same also applies to supply and discharge lines which end at the disposable fermenter or disposable reactor or are lead out of the disposable fermenter or disposable reactor.

One or more sensors integrated into the disposable container may be sterilized together with disposable container. As a result of the sterilization, and/or in the event that there is a longer time span between the sterilization and the placement of the disposable containers and the integrated sensors into operation, properties of the integrated sensors can change, which can lead to a change in the respective characteristic sensor curves, for example to a zero point drift. Potentiometric and amperometric sensors often comprise membranes which should ideally be stored moist. The moist storage ensures that the sensor outputs reliable measured values immediately as of being placed in operation.

Given electrochemical sensors, a sensor arrangement in the sense of the present specification, a measurement of the electrical voltage or of the electrical current between two electrodes takes place. In general, an electrode is also called a “terminal lead”. An electrical potential is created via the accumulation, incorporation, or electrochemical conversion of the ions, atoms, or molecules to be measured at the sensitive region. A reference electrode, which provides a fixed potential reference point, is necessary for measuring the electrical voltage. The reference electrode consists of the combination of a metal with a low-solubility salt of this metal and an electrolyte with a fixed concentration of the anion of the salt and, for the measurement, should have a good electrical connection to the measuring medium, i.e., a high ion conductivity. Since the ion composition of the reference electrode determines the reference potential, the ion concentration in the reference electrode must not change. The connection of the reference electrode to the measuring medium takes place via a semipermeable connection, generally a transport, for example a diaphragm, which slows the ion exchange and thus enables a reference potential that is stable over a longer period of time.

Transports, such as diaphragms, are produced from a wide variety of materials, such as porous ceramics, plastics, arrays, or microchannel plates. Material and size are thereby adapted to the respective application; large-area and coarse-pore diaphragms are used given highly contaminated measuring media to prevent blockage of the diaphragm. Small and fine-pore diaphragms, on the other hand, more strongly slow down a penetration of foreign ions as well as the diffusion of the ions present in the reference electrode, and increase the service life of the reference electrode.

If stored in air, the reference electrolyte evaporates from the diaphragm and a salt crust forms; although the use a gel electrolyte and/or covering the diaphragm can reduce the formation of the salt layer, it cannot completely prevent it. Upon being placed in operation, the salt layer must be dissolved and the diaphragm must be completely moistened again before a reliable measurement is possible. Alternatively, the storage of electrochemical liquid sensors takes place in an electrolyte that has the same composition as the reference electrolyte.

The sensitive region is also preferably stored in a liquid if the sensor is not used. Electrochemical sensors stored in liquid are more quickly ready for use than those stored dry. Before the measurement, however, a calibration must always take place in a liquid having a defined concentration of the ions, atoms, or molecules to be measured.

Independent of the storage, the transfer of the electrochemical sensor from the storage medium into the calibration medium and subsequently into the measuring medium is complicated, given sterile or inert measuring conditions. This requires either the introduction of calibration solution into the measuring system or a transfer of the calibration solution into or from additional calibration chambers, wherein a carryover of the calibration electrolyte into the measuring medium can be precluded only with great technical effort. Sensors stored dry must subsequently be stored in a sterile liquid before being introduced into a process, whereby a high response time results. The mentioned procedures are time-consuming, error-prone, and associated with high costs.

DE 10 2016 101 715 A1 discloses a sensor arrangement comprising a housing which can be connected to a process container and in which a guide channel is formed, and a sensor body which can be moved in the guide channel in the axial direction between a first position and a second position and has a sensor element which can be extended out of the housing and serves to detect a measured quantity of a measuring medium, wherein an end portion of the sensor body has an end face base surface and a peripheral surface, wherein the sensor element forms a part of the peripheral surface. The measuring cell body comprises a measuring half-cell and a reference half-cell, wherein the body is movable between the two positions.

US 2020/0217817 A1 discloses a sensor with a sensor element which is held in a storage space filled with a storage medium, wherein the storage medium can also be used as a calibration medium. The sensor element can have a sensor surface which is remote from the distal end of the sensor element, so that an inactive portion of the sensor element can interact with a sealing element, such as an O-ring, in order to form a part of the seal that holds the storage medium/calibration medium. The sensor element can be driven out of the storage space and be retracted in order to expose the sensor surface to a measuring medium, whereas the storage medium is stored in the storage space for the validation after the measurement. The sensor also comprises a reference half-cell element with a liquid passage, wherein the reference half-cell element is designed such that it moves together with the sensor element so that, when the sensor surface is exposed to the storage medium/calibration medium, this is also true for the liquid passage. If the sensor surface is exposed to a measuring medium, the liquid connection is also exposed to the measuring medium.

However, an uncomplicated use of the sensor in sterile applications is not possible in this way.

### SUMMARY

The present disclosure is therefore based on the object of simplifying the process for placing sensors in operation in sterile fields of application.

The object is achieved by a sensor arrangement comprising a storage chamber comprising an interior space which contains a reference/storage/calibration liquid, with an opening; and a reference terminal lead which contacts the reference/storage/calibration liquid and can be connected to a superordinate unit; and a sensor tube comprising a sensitive region for detecting a measured quantity of the measuring medium; and a measuring terminal lead; wherein the sensitive region can be electrically connected to the superordinate unit via the measuring terminal lead; wherein the sensor tube is movably arranged in the opening of the storage chamber and can be moved from a first position to a second position, wherein the sensitive region is arranged on/in the sensor tube such that, in the first position, it is located in the interior space of the storage chamber and, in the second position, is located outside the storage chamber; wherein the storage chamber, the opening, and the sensor tube are designed such that, in the first position, the reference/storage/calibration liquid is prevented from escaping from the interior space; and wherein the storage chamber, the opening, and the sensor tube are designed such that, in the second position, a liquid transport is formed.

A sensor arrangement thus results with a sensor tube and a sensitive region that can be stored over a longer period of time. The arrangement can be sterilized, for instance gamma-sterilized, and finally easily calibrated. Nevertheless, a simple possibility results to move the sensitive region into a position in which it is in contact with the medium to be measured.

In the first position, an escape of the reference/storage/calibration liquid from the interior space is prevented; the sensitive region is thus stored moist. In one embodiment, the opening comprises one or more seals for this purpose. In one embodiment, the outer diameter of the sensor tube is adapted to the diameter of the opening.

A calibration can take place in the first position.

A liquid transport is formed in the second position. The liquid transport forms a fluid channel from the interior space of the storage chamber to the outside, i.e., to a container connected to the sensor arrangement. In one embodiment, this container is a measuring cell; see below. The liquid transport then results in a measuring path via the sensitive element or the measuring terminal lead.

One embodiment provides that the storage chamber is arranged coaxially around the sensor tube.

One embodiment provides that the sensor arrangement comprises a measuring cell for receiving measuring medium, wherein the measuring cell is connected to the storage chamber via the opening and wherein, in the second position, the sensitive region is located in the measuring cell.

One embodiment provides that the measuring cell and the storage chamber are designed as one piece.

One embodiment provides that the sensor arrangement comprises a transport-forming element which is arranged such that, in the second position of the sensor tube, the liquid transport is designed as a liquid transport filled with reference/storage/calibration liquid. The transport-forming element is arranged such that, in the first position of the sensor tube, the transport-forming element is arranged in the storage chamber.

One embodiment provides that the transport-forming element is arranged on or at the sensor tube.

One embodiment provides that the transport-forming element is designed as a component, such as an annular diaphragm.

One embodiment provides that the transport-forming element is designed as a surface texture of the sensor tube, such as one or more axially arranged slits, roughing, or tapering.

One embodiment provides that a seal, such as an O-ring, which seals the storage chamber in the first position is arranged in the opening.

One embodiment provides that the transport-forming element is arranged annularly in the opening, and that the sensor tube comprises, at the measuring cell-side end, a plate seal which radially surrounds the transport-forming element and, in the first position, seals the storage chamber with respect to the measuring cell.

One embodiment provides that the measuring cell comprises a second opening and the transport-forming element is arranged in the second opening, and the sensor tube on the measuring cell-side end comprises a plate seal which radially surrounds the transport-forming element and, in the first position, seals the storage chamber with respect to the measuring cell.

One embodiment provides that the storage chamber comprises one or more filling openings for reference/storage/calibration liquid.

One embodiment provides that the reference/storage/calibration liquid is a liquid with a defined anion content (e.g., 3 mol/l chloride ions) in order to form a stable reference electrode. The pH value is adjusted for calibration by a preferably inorganic pH buffer. The liquid prevents leaching of the swelling layer during storage, in one embodiment by Li or Na ions. In one embodiment, the reference/storage/calibration liquid is an internal buffer of a normal pH electrode.

One embodiment provides that a temperature sensor is arranged in the sensor tube.

One embodiment provides that the sensor arrangement comprising: a housing that is in mechanical contact with the sensor tube, and a movement of the housing causes a movement of the sensor tube.

One embodiment provides that the housing is arranged like a sleeve around the storage chamber.

One embodiment provides that the sensor arrangement is designed as a potentiometric sensor arrangement with the sensor tube, such as in the measuring cell, as a measuring half-cell and the storage chamber as a reference half-cell.

One embodiment provides that the sensitive region is designed as an ion-selective membrane, such as a pH-sensitive membrane. The ion-selective membrane is designed to determine the concentration or activity of a particular type of ion.

One embodiment provides that the membrane is designed as a glass membrane, such as a dome.

One embodiment provides that the membrane is designed as an enamel layer.

One embodiment provides that the housing comprises a plug head, wherein the measuring terminal lead and the reference terminal lead are connected to the plug head.

One embodiment provides that the reference/storage/calibration liquid is formed from an aqueous electrolyte solution.

One embodiment provides that the electrolyte solution contains, in a predetermined activity or concentration, an analyte that correlates with the measured quantity.

One embodiment provides that the electrolyte solution contains ions, preferably chloride ions, forming the potential of the reference terminal lead.

The object is further achieved by a method for placing a sensor arrangement into operation as described above, comprising the steps: sterilizing the sensor arrangement, for instance with ionizing radiation, such as gamma or beta radiation. A calibration, optionally also an adjustment, of the sensor arrangement then takes place, whereby changes to the sensor arrangement due the sterilization are calibrated/adjusted out. The movement of the sensor tube from the first to the second position then takes place. The liquid transport thereby forms. In the next step, the measurement can take place.

In one embodiment, the sensor arrangement is attached to the container with the liquid to be measured. Depending on the type of application, this step takes place before or after the sterilization, or before or after the calibration.

In summary, the present document discloses a sensor arrangement with a storage chamber, a sensor tube, and a measuring cell. The sensor tube is arranged such that it can be introduced into a process by an operator. The storage chamber comprises a reference terminal lead and is filled with a liquid which can also be used as a reference liquid and, by defined addition of the ions, atoms, or molecules to be measured, also as a calibration electrolyte. The filling of the storage chamber can be realized via corresponding openings. The storage chamber comprises an opening for receiving the sensor tube. In order to avoid leakage or to prevent the liquid from escaping from the storage chamber, one or more sealing element(s) can be used. The sensor tube has a sensitive region to which a terminal lead is connected. The transport required for the contact between the measuring medium and the reference terminal lead can be integrated in/on the sensor tube. In the first position, the sensitive region of the measuring tube is located entirely in the storage chamber. A closed circuit exists between reference terminal lead and sensitive region, and the voltage can be measured, or regulated for a current measurement. Since a defined concentration of the ions, atoms, or molecules to be measured is present in the reference electrolyte, a calibration of the measured quantity can be performed. Via a displacement of the sensor tube from the storage chamber into the measuring cell by a defined distance, the sensitive region is introduced into the measuring medium and the transport-forming element is placed such that it enables the contact of the reference electrolyte to the measuring medium via the sealed opening (second position). The measuring medium can be conducted via inlet and outlet channels through the measuring cell to the sensitive region, or the sensor tube dips directly into a chamber containing the measuring medium. All hose connections on the sensor arrangement can be sealed before a sterilization. The sterile connection can thus be maintained during the entire process implementation and in the process. Via the combined use of the storage chamber for storing and for calibrating the measuring tube, as well as a transport, for example one attached to the sensor tube, no additional electrolytes are needed for calibration. Via the permanent moisture storage with exclusion of oxygen, salt is prevented from forming on the transport and the sensitive region is prevented from drying out. Via the closed structure, the claimed sensor arrangement can be sterilized by ionizing radiation, such as electron beams, gamma radiation, or x-ray radiation; stored; and installed in plants while maintaining sterility.

### BRIEF DESCRIPTION OF THE DRAWINGS

This is explained in more detail with reference to the following Figures.

FIG. 1a/b show the claimed sensor arrangement in a first or second position.

FIG. 2 shows the claimed sensor arrangement in one embodiment in the second position.

FIG. 3a-d show the claimed sensor arrangement in one embodiment in a first or second position, with a detailed view.

FIG. 4a/b how the claimed sensor arrangement in one embodiment in a first or second position.

FIG. 5a/b show the claimed sensor arrangement in one embodiment in a first or second position.

In Figures, the same features are labeled with the same reference signs.

### DETAILED DESCRIPTION

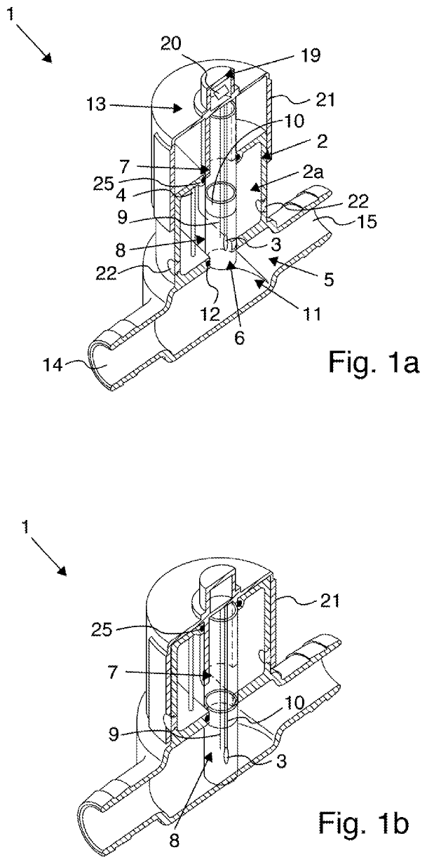

The claimed sensor arrangement in its entirety bears the reference sign 1 and is shown in a first position in FIG. 1a.

FIG. 1a and FIG. 1b show a first example of a sensor arrangement 1, for example with a housing 13 formed from a plastic with hygiene approval. The sensor arrangement 1 can be connected to a process container, such as a process container in disposable technology, for example a fermenter, bag, pouch, sac, chamber, container, a hose or pipeline with a rigid or flexible wall made of a plastic with hygiene approval, or the like. An embodiment for connecting to a hose or pipeline is shown. The process container is referred to below as a measuring cell 5. The measuring cell 5 comprises an inlet 14 and an outlet 15. In the embodiment for another container, the sensor arrangement 1 then has a connecting strip (not shown; as a type of flange) which can be glued or welded to the wall of the process container. Accordingly, the sensor arrangement 1 then does not comprise an inlet 14 and outlet 15; rather, the sensor arrangement 1 is adapted to the container to be connected.

A plastic, e.g., PE, PPSU, PVDF, or PEEK, for example, is considered as a material for the measuring cell 5.

The sensor arrangement 1 comprises a storage chamber 2 with an interior space 2a which is designed to receive a reference/storage/calibration liquid, and a reference terminal lead 4 which can be connected to a superordinate unit (not shown). The storage chamber 2 has, for example, a circular cylindrical shape with. In one embodiment, the storage chamber 2 has an elliptical or polygonal base surface, for instance has a quadrangular or pentagonal design. The storage chamber 2 comprises one or more filling openings 22 for reference/storage/calibration liquid.

In the embodiment in FIG. 1a, the sensor arrangement 1 comprises a measuring cell 5. The storage chamber comprises an opening 6, such as a circular opening. The measuring cell 5 and the storage chamber 2 are, for example, manufactured in one piece, wherein they are connected to one another via the opening 6. Alternatively, the two can be manufactured from separate parts and be connected to one another accordingly, for instance by joining, gluing, or welding. The measuring cell 5 is designed to receive measuring medium 11 via the inlet and outlet 14, 15 (see above). The measuring cell 5 is, for example, a flow cell which can be introduced into a line system. This is shown in FIG. 1a/b.

A sensor tube 7, for example a cylindrical sensor tube, is mounted in an axially movable manner in the opening 6, i.e., the opening 6 forms a guide channel. The sensor tube 7 is movable at least from a first position (FIG. la, storage position) into a second position (FIG. 1b, measurement position). The storage chamber 2 is thus arranged coaxially around the sensor tube 7. In one embodiment, the opening 6 is arranged off-axis, i.e., is displaced from the main axis of the storage chamber. This can be the case given an elliptical basic shape.

The sensor arrangement 1 is preferably designed as a single-use sensor, i.e., the sensor tube 7 can be displaced only once from the first into the second position. The sensor tube 7 comprises a sensitive region 8 for detecting a measured quantity of the measuring medium 11, and a measuring terminal lead 9, wherein the sensitive region 8 can be connected to the superordinate unit (not shown) via the measuring terminal lead 9. In the first position, the sensitive region 8 is located in the storage chamber 2 and, in the second position, it is located in the measuring cell 5. See below for details regarding the measurement.

The storage chamber 2, the opening 6, and the sensor tube 7 are designed such that, in the first position, the reference/storage/calibration liquid is prevented from escaping from the interior space 2a. The storage chamber 2, the opening 6, and the sensor tube 7 are designed such that a liquid transport is formed in the second position. This is explained below.

For this purpose, the sensor arrangement 1 comprises, for instance, a transport-forming element 10, 16, 26 which is arranged such that, in the second position of the sensor tube 7, a liquid connection exists between the interior space 2a of the storage chamber 2 and the outside, i.e., for instance, to the measuring cell 5. The transport-forming element 10, 16, 26 is arranged such that, in the second position of the sensor tube 7, the liquid transport is designed as a liquid transport filled with reference/storage/calibration liquid. A fluid channel from the interior space 2a to the outside is thus formed. The transport-forming element 10 is often referred to as a “liquid”. The transport-forming element 10 is arranged such that, in the first position of the sensor tube 7, the transport-forming element 10 is arranged in the storage chamber 2.

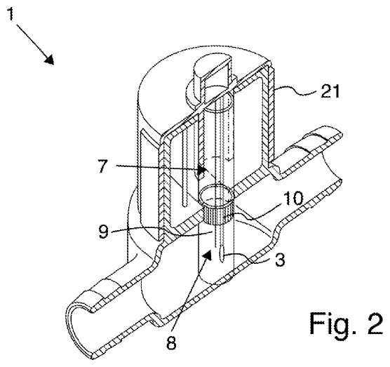

FIG. 1a and FIG. 1b show an embodiment in which the transport-forming element 10 is arranged on or at the sensor tube 7, and in fact as a component (i.e., as an interchangeable element), such as an annular diaphragm. FIG. 2 shows an embodiment of the transport-forming element 10 as a surface texture of the sensor tube 7, for instance as one or more axially arranged slits, roughings, or taperings. FIG. 2 shows the second position.

The storage chamber 2, the measuring cell 5, and the sensor tube 7 are designed such that, in the first position, the storage chamber 2 is sealed with respect to the measuring cell 5. The present gaps are filled or evened out by sealing elements 12. A hermetic sealing of the two chambers 2, 5 is generated by the sealing elements 12 and the sensor tube 7. During the axial movement of the sensor tube 7, the seal is completely maintained, so that no exchange of fluids takes place between the chambers 2, 5.

The storage chamber 2 is closed after its filling. A fluid is contained therein, which serves for the moist storage of the sensitive region 8 and of the transport-forming element 10. As mentioned, the reference terminal lead 4 is located in the storage chamber 2. Via the known pH value of the storage fluid, a calibration can be performed in the first position (storage position) before placement into service. A closed measuring circuit is present in the storage position (i.e., the first position) since reference terminal lead 4 and sensitive region 8 are located in the same medium.

Upon being placed into service, the sensor tube 7 is moved into the measuring cell 5 such that the sensitive region 8 is located entirely in the measuring cell 5, and such that regions of the transport-forming element 10 are located both in the storage chamber 2 and in the measuring cell 5. If measuring medium 11 is guided through the measuring cell 5 (via inlet 14 and outlet 15), it contacts the sensitive region 8 and the transport-forming element 10 on the side of the measuring cell 5, wherein a reference solution contacts the reference terminal lead 4 and the transport-forming element 10 on the side of the storage chamber 2. The storage chamber 2 does not move. The reference terminal lead 4 remains in the storage chamber 2 and does not move.

A displacement mimic, which is connected to the sensor tube 7, serves to move the sensor tube 7. The displacement mimic comprises a sleeve bushing 21, which is rigidly connected to the sensor tube 7 so that an axial movement of the sleeve bushing 21 toward the measuring medium 11 causes an axial movement of the sensor tube 7 in the direction of the measuring medium 11.

As mentioned, the sensor arrangement 1 comprises a housing 13, wherein the housing 13 comprises the sleeve bushing 21. An axial movement of the housing 13 thus causes a movement of the sensor tube 7. A plastic, for instance PC, COC, PE, PPSU, PVDF, or PEEK, for example, is considered as a material for the housing 13.

In the first position, the sensor tube 7 also projects from the storage chamber 2 at the upper end thereof (i.e., toward the displacement mimic). In order that no liquid flows out, the storage chamber 2 comprises one or more corresponding seals 25.

The two end positions that can be reached by moving the sensor tube 7 are shown in FIG. 1a and FIG. 1b. In FIG. 1a, the sensor tube 7 is retracted completely into the storage chamber 2 in the first position so that only its front-side base surface is still in contact with the environment. In FIG. 1b, in the second position, a portion of the sensor tube 7, primarily the sensitive region 8, is extended out of the storage chamber 2. Both end positions can be predetermined in a manner known per se by stops formed in the displacement mimic, the housing 2, the sleeve 21, and/or the sensor tube 7 5. An end position latching or end position fixing can also be provided in a manner familiar to the person skilled in the art.

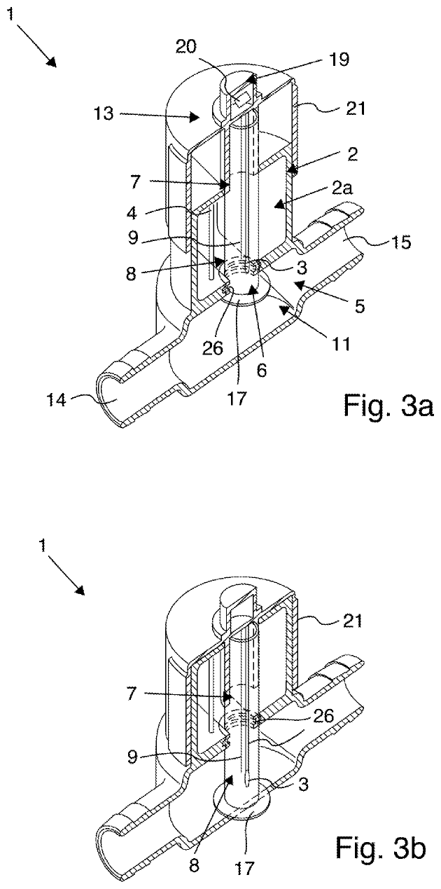

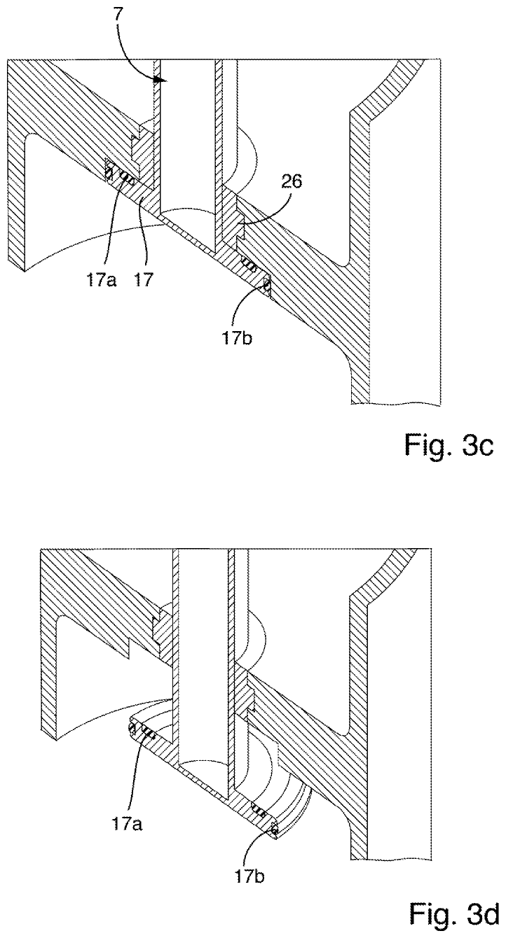

FIG. 3a and FIG. 3b show an embodiment of the sensor arrangement in the first and second position, respectively. The transport-forming element 26 is thereby arranged annularly in the opening 6, and the sensor tube 7 comprises at the measuring cell-side end a plate seal 17 with an axially or radially arranged sealing element 17a, 17b (FIG. 3c and FIG. 3d show both embodiments, wherein only one sealing element 17a, 17b can also be used), wherein the plate seal 17 radially surrounds the transport-forming element 26 and seals the storage chamber 2 with respect to the measuring cell 5 in the first position, and exposes the diaphragm to the measuring medium in the second position. FIG. 3c and FIG. 3d show this in detail.

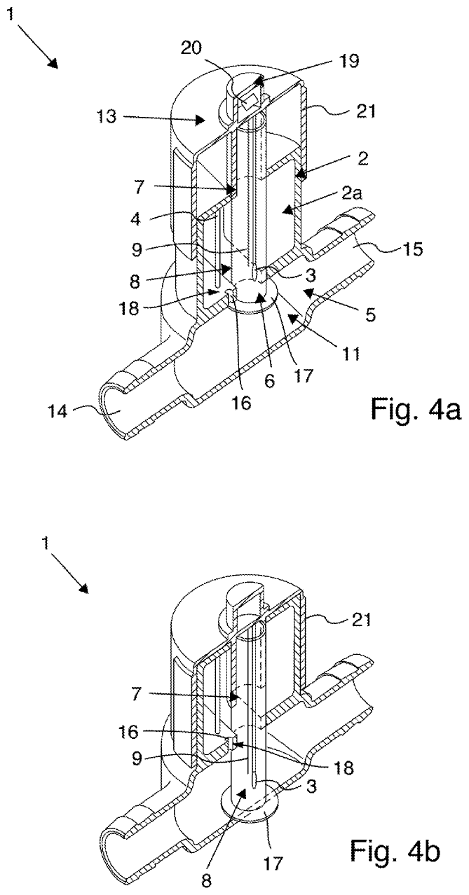

FIG. 4a or FIG. 4b show an embodiment of the sensor arrangement in the first and second position, respectively. The measuring cell 5 thereby comprises a second opening 18, and the transport-forming element 16 is arranged in the second opening. The sensor tube 7 comprises at the measuring cell-side end a plate seal 17, which radially surrounds the transport-forming element 16 and seals the storage chamber 2 with respect to the measuring cell 5 in the first position.

As mentioned, the transport-forming element 10, 16, 26 establishes the electrical connection to the reference cell upon movement of the sensor tube 7. The transport, i.e., the liquid transport in the wording of the claim, is formed after displacement of the sensor tube 7 into the second position. The transport-forming element 10, 16, 26 is thereby the prerequisite for the function of the storage chamber as a reference cell. Various embodiments of the transport-forming element 10, 16, 26 are possible:

constituent of the movable sensor element

as a ring element, for instance as a diaphragm (embodiment as a part or component, e.g., FIG. 1a/b)

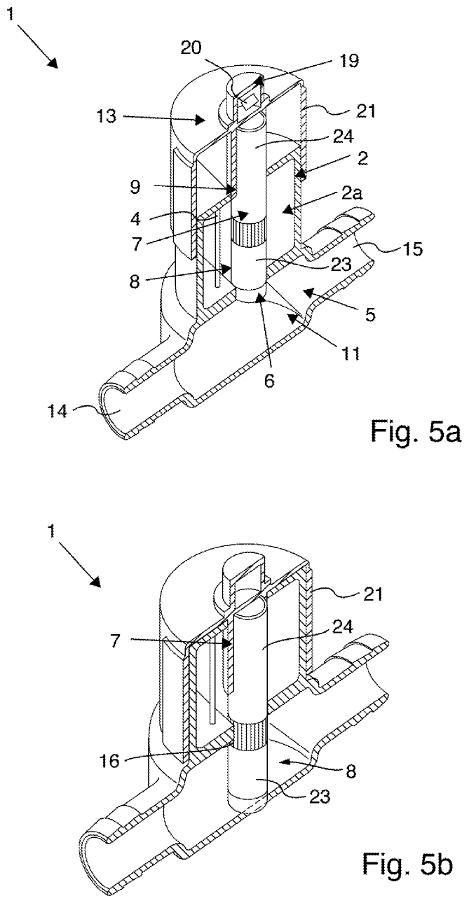

as a surface texture (embodiment as a functional surface, e.g., FIG. 2, FIG. 5a/b)

constituent of the complete assembly (embodiment as a component)

with contacting the sensor tube (e.g., FIG. 3a/b)

without contacting the sensor tube (e.g., FIG. 4a/b)

A liquid of known composition in the storage chamber 2 serves for the moist storage of the sensor tube 7 stored therein (first position; of the sensitive region 8 and of the transport-forming element 10). The same liquid serves for the calibration of the sensor before placement into operation, and the same liquid serves for referencing during the measurement. The liquid remains in the storage chamber 2, whereas the sensor tube 5 can be moved into the measuring cell 5.

In the exemplary embodiments illustrated here, the sensor arrangement 1 comprises a potentiometric sensor with a pH measuring half-cell and a reference half-cell.

The measuring half-cell is formed by the measuring cell 5 with the sensor tube 7 in the second position. For this purpose, the sensor tube 7 comprises the sensitive region 8. In one embodiment, the sensitive region 8 is designed as an ion-selective membrane, such as a pH-sensitive membrane. The membrane is a glass membrane. The sensitive region 8 can thereby also be designed as a cap.

The measuring terminal lead 9, which is in electrical contact with the sensitive region 8, is located in the interior of the sensor tube 7. The interior of the sensor tube 7 forms the measuring half-cell chamber, wherein a liquid or gel-like internal electrolyte can be received therein. In the present example, the internal electrolyte is a buffer solution with a predetermined chloride concentration. The measuring terminal lead 9 contacts the internal electrolyte or the electrically conductive inner surface of the measuring half-cell and is electrically conductively connected to a contact point outside the measuring half-cell chamber (not illustrated in Figures; a superordinate unit, for instance). The measuring terminal lead 9 can be a metal wire, for example a chlorided silver wire.

The measuring half-cell chamber, i.e., the sensor tube 7, is closed on the rear side, for example by means of a plastic casting compound or by fusing or gluing.

In one embodiment of the sensitive region 8, the sensitive region is designed as a layer which rests on the electrically conductive terminal lead. In this embodiment, the terminal lead is designed as a solid terminal lead. The layer may be an ion-sensitive enamel layer 23, which is shown in FIG. 5a and FIG. 5b in the first and second positions and is explained below:

On its outside, the sensor tube 7 is covered with a system of layers. In this exemplary embodiment, the sensor tube 7 has a base layer 24 of an insulating material, for example an insulating enamel layer, on the front side. An ion-selective enamel layer 23, which in the present example comprises pH glass, is arranged above the base layer. On the rear side, the ion-selective layer is electrically contacted by a metallic terminal lead. The discharge takes place in the longitudinal direction, for example along the outer lateral surface of the sensor tube 7 up to the end face with the back surface of the sensor tube 7 opposite the ion-selective layer 23. The terminal lead is embedded in an electrically insulating coating, for example an insulating enamel layer, which electrically insulates the terminal lead from the sensor tube 7 and from the environment of the measuring half-cell. The coating may be formed from a plurality of individual layers of identical or different glass compositions. The terminal lead may, for example, be designed as a metallic coating on a layer of the coating.

In a modification of the exemplary embodiment, the sensor tube 7 can be designed to be electrically conductive, for instance metallic, and then itself serve as a terminal lead. In this instance, the ion-selective enamel layer is applied directly to the sensor tube 7. A surface region not covered by the ion-selective enamel layer 23, for example the lateral surface of the sensor tube 7, may be covered by an electrically insulating coating, for example an insulating enamel, and thus be insulated from the environment of the measuring half-cell.

“Enamel electrodes” normally have a metallic base body to which an ion-selective, such as a pH-selective, glass layer 23 is applied. The ion-selective layer may be an enamel coating.

According to the definitions/labeling standards, RAL registration RAL-RG 529 A2 from July 2007 by RAL Deutsches Institut fur Güatesicherung and Kennzeichnung e. V. [RAL German Institute for Quality Assurance and Certification, registered association], a vitreous material that is produced by completely or partially melting substantially oxidic raw materials is referred to as an enamel. The inorganic preparation thus produced is applied with additives in one or more layers to workpieces made of metal or glass and fused at temperatures above 480° C. Base constituents of (ion-selective) enamel layers are, for example, one or more of the oxides silicon oxide, sodium oxide, potassium oxide, calcium oxide, magnesium oxide, and aluminum oxide. An ion-selective glass, e.g., pH glass, applied to a metallic base body using such a method is therefore also referred to hereinafter as an ion-selective enamel layer or, in the case of an enamel layer specifically selective for hydronium ions, as a pH enamel layer, and a corresponding electrode as an enamel electrode. In this exemplary embodiment, no internal electrolyte is used.

In each of the above-described embodiments, outside the measuring half-cell chamber and the reference half-cell chamber (see below), a measuring circuit 20 can be arranged in the housing 13, which measuring circuit is electrically conductively connected to the measuring terminal lead 9 and is designed to detect a potential difference between the measuring terminal lead 9 and the reference terminal lead 4 (not shown in Figures). By means of a plug connection, for instance a galvanically isolating connection, for example an inductive connection, or a contact plug connection, between a plug head 19 connected to the housing 13 and a complementary counterpart (not shown in the Fig.), the measuring circuit can be designed to be connected to a superordinate electronic data processing device for transmitting measurement signals and/or data.

The reference half-cell is formed by the storage chamber 2 (which forms the reference half-cell chamber) with the reference terminal lead 4. The reference half-cell chamber contains the reference/storage/calibration liquid, such as potassium chloride or sodium chloride. In order to combine all functions, the reference/storage/calibration liquid must have a defined anion concentration for the reference electrode (e.g., 3 mol/l Cl—), a defined and stable pH value (calibration), and a composition (storage) that is advantageous for maintaining the swelling layer. Arranged in the storage chamber 2 is the reference terminal lead 4, for example a chlorided silver wire, which contacts the reference/storage/calibration liquid and is electrically conductively connected to a further contact point outside the reference half-cell chamber (not shown in Figures).

The reference half-cell chamber is closed on the rear side, for example by means of a plastic casting compound or by fusing or gluing.

The sensor arrangement 1 comprises a temperature sensor 3 which is arranged, for example, in the sensor tube 7. The temperature sensor 3 is electrically connected to the measuring circuit 20.

1. A sensor arrangement, comprising
a storage chamber, comprising
an interior space that contains a reference/storage/calibration liquid, with an opening, and
a reference terminal lead which contacts the reference/storage/calibration liquid and can be connected to a superordinate unit, and
a sensor tube, comprising
a sensitive region for detecting a measured quantity of the measuring medium, and
a measuring terminal lead, wherein the sensitive region can be electrically connected to the superordinate unit via the measuring terminal lead,
wherein the sensor tube is movably arranged in the opening of the storage chamber and can be moved from a first position to a second position,
wherein the sensitive region is arranged on/in the sensor tube such that, in the first position, it is located in the interior space of the storage chamber and, in the second position, it is located outside the storage chamber,
wherein the storage chamber, the opening, and the sensor tube are designed such that, in the first position, the reference/storage liquid is prevented from escaping from the interior space, and
wherein the storage chamber, the opening, and the sensor tube are designed such that a liquid transport is formed in the second position.

2. The sensor arrangement of claim 1,
wherein the storage chamber is arranged coaxially around the sensor tube.

3. The sensor arrangement of claim 1, comprising
a measuring cell for receiving measuring medium, wherein the measuring cell is connected to the storage chamber via the opening, and wherein, in the second position, the sensitive region is located in the measuring cell.

4. The sensor arrangement of claim 3,
wherein the measuring cell and the storage chamber are designed in one piece.

5. The sensor arrangement of claim 1, comprising
a transport-forming element which is arranged such that, in the second position of the sensor tube, the liquid transport is designed as a liquid transport filled with reference/storage/calibration liquid.

6. The sensor arrangement of claim 5,
wherein the transport-forming element is arranged on or at the sensor tube.

7. The sensor arrangement of claim 6,
wherein the transport-forming element is designed as an annular diaphragm.

8. The sensor arrangement of claim 6,
wherein the transport-forming element is designed as a surface texture of the sensor tube, such as one or more axially arranged slits, roughing, or tapering.

9. The sensor arrangement of claim 1,
wherein a seal is arranged in the opening, which seal seals off the storage chamber in the first position.

10. The sensor arrangement of claim 5,
wherein the transport-forming element is arranged annularly in the opening, and the sensor tube at an end region comprises a plate seal which radially surrounds the transport-forming element and seals off the storage chamber in the first position.

11. The sensor arrangement of claim 5,
wherein the measuring cell comprises a second opening and the transport-forming element is arranged in the second opening, and the sensor tube at an end region comprises a plate seal which radially surrounds the transport-forming element and seals off the storage chamber in the first position.

12. The sensor arrangement of claim 1,
wherein the storage chamber comprises one or more filling openings for reference/storage/calibration liquid.

13. The sensor arrangement of claim 1,
a housing that is in mechanical contact with the sensor tube, and a movement of the housing causes a movement of the sensor tube.

14. The sensor arrangement of claim 1,
wherein the housing is arranged like a sleeve around the storage chamber.

15. The sensor arrangement of claim 1,
wherein the sensor arrangement is designed as a potentiometric sensor arrangement with the sensor tube as a measuring half-cell and the storage chamber as a reference half-cell.

16. The sensor arrangement of claim 1,
wherein the sensitive region is designed as an ion-selective membrane.

17. The sensor arrangement of claim 16,
wherein the membrane is designed as a glass membrane.

18. The sensor arrangement of claim 16,
wherein the membrane is designed as an enamel layer.

19. The sensor arrangement of claim 1,
wherein the reference/storage/calibration liquid is formed from an aqueous electrolyte solution.

20. The sensor arrangement of claim 1,
wherein the electrolyte solution contains, in a predetermined activity or concentration, an analyte that correlates with the measured quantity.

21. The sensor arrangement according to claim 1, wherein the electrolyte solution contains ions forming the potential of the reference terminal lead.

22. A method for placing a sensor arrangement into operation, comprising the steps of:
sterilizing the sensor arrangement,
calibrating the sensor arrangement, and
moving the sensor tube from the first position into the second position.
